(19) United States
(12) Patent Application Publication
(10) Pub. No.: US 2024/0049748 A1
(43) Pub. Date: Feb. 15, 2024

(54) FOAMED, RESILIENT, PROTEIN-BASED PRODUCT

(71) Applicants: Erich WINDHAB, Hemishofen (CH); Joel ZINK, Zurich (CH); Cédric SAX, Zurich (CH); Bhaskar MITRA, Kolkata (IN)

(72) Inventors: Erich WINDHAB, Hemishofen (CH); Joel ZINK, Zurich (CH); Cédric SAX, Zurich (CH); Bhaskar MITRA, Kolkata (IN)

(73) Assignee: ETH ZÜRICH, Zurich (CH)

(21) Appl. No.: 18/259,257

(22) PCT Filed: Dec. 6, 2021
(86) PCT No.: PCT/EP2021/000154

§ 371 Date: Jun. 23, 2023

(30) Foreign Application Priority Data
Dec. 23, 2020 (DE) 10 2020 007 888.5

(51) Int. Cl. A23J 3/22; A23J 3/14; A23J 3/26; A61K 47/42

(52) U.S. Cl. CPC A23J 3/227; A23J 3/14; A23J 3/26; A61K 47/42

(57) ABSTRACT

The invention relates to a product having a foam structure with foam pores which are partially open to the surface and are filled, partially or completely, with a fluid. The invention further relates to a method having embodiments for filling open foam pores in a defined manner. The invention also relates to a device having embodiments for filling open foam pores in a defined manner. The invention furthermore relates to the use of the foam products designed and filled according to the invention as main components for plant protein-based meat analogs. Particular advantages of the invention are the simplified multifunctionality of the products according to the invention in terms of their sensory and nutritional properties for adaptation to preferences and needs of certain target groups including individual customization.

### FIELD

The invention relates to a foamed, resilient, protein-based product.

Furthermore, the invention relates to a method with method variants for producing such a product set with a defined degree of pore filling.

The invention also relates to a device with device variants for carrying out the method according to the invention.

Finally, the invention relates to a use of the product according to the invention as a food product that can be customized in terms of its sensory, nutritional and health-promoting functionalities, in particular on the basis of plant-protein-based meat analogs.

### Prior Art

Foamed, open-pored (spongy) product systems are known in the food sector for baked goods or in the field of instant products (instant soups, drinks, sauces) /1/. For the latter, the wetting and penetrating behavior in contact with fluids as well as the rapid and complete dispersibility, possibly coupled with rapid dissolving, are crucial quality and convenience criteria /2/. For the first-mentioned category of baked goods, wetting and partial dispersion occur in the saliva or liquid supplied to the consumer's oral cavity.

Foamed food systems from dry extrusion methods are known in particular as starch-based snack foods. They have low water contents (approx. ≤3-5%) to ensure their crispiness. In the extrusion methods used for such products, foam formation takes place through steam expansion as a result of a sudden drop in static pressure at the extruder nozzle outlet. Due to its fast kinetics, this process can only be controlled within wide limits with regard to the setting of a foam structure. As a result, the resulting products usually have very large pores and mostly open pores /3/.

In a few recent developments, extrusion-based foaming processes with controlled adjustment of the resulting foam structure have been implemented. In these cases, the foam was generated in the usually highly viscous masses by gas dissolution and subsequent foam bubble nucleation and foam bubble growth under controlled pressure release. The process development for doughs carried out in this way is considered wet extrusion, but under “low temperature conditions” of approx. 40-60° C. /4, 5/.

More recently, the foaming of plant protein-based meat analogs was achieved using a further developed method variant of “High Moisture Extrusion Cooking (HMEC), which takes place at high static pressures of up to 80 bar and temperatures of up to 170° C. There are no publications on this topic yet.

Reference is made below to such foam products, which can have open and closed foam pores in different ratios.

So far, the problem of a targeted filling of foamed, partly to fully open-pored food systems has not been addressed according to the published state of knowledge. This is due to the fact that the few known open-pore food products such as baked goods or meringue-like marshmallows cannot be wetted with aqueous fluids without disintegrating, and oil-based fluids neither wet well nor are nutritionally or culinary relevant in this context. The novel, foamed, plant-protein-based meat analogs produced using HMECF technology represent something special with regard to the high bound water fraction of up to >60%. Since no free water separates in the foam pores as a result of the strong molecular or intermolecular water binding in the denatured, folded protein structure, the gas pore spaces created can be used to introduce further liquids into them. This can in principle occur at least in part via long-term (hours to days) immersion of such foamed products in liquids and fluid-gas exchange taking place by diffusion. This would correspond to a kind of “marinating process”. However, such an approach leaves little room for (i) industrial production with (ii) diverse variations for product personalization and in particular (iii) the integration of sensory and nutritional product functionalities.

The possibility of accelerated absorption of additional fluid fractions and their capillary fixation in the meat analogs already produced with a pronounced water content based on HMEC is given by their foaming and adjustment of the open porosity. This poses the task of creating new sensory and nutritional tailor-made products in a form that has not been possible up to now.

The publication “Albert Schweitzer Foundation: Vegane Großverpflegung-ein Leitfaden, 2. edition, as of May 2017” describes vegan large-scale catering, i.e. a guide for large kitchens with various recipes.

WO 2012/158023 A1 describes a method for producing a structured plant protein extrudate, comprising the steps of
(a) providing an aqueous protein composition containing plant protein, wherein the protein content, based on dry matter, is below 90% by weight;
(b) subjecting the aqueous protein composition to one or more kneading steps to form a dough;
(c) heating the dough to a temperature above the denaturation temperature of the protein;
(d) subjecting the dough to shear and pressure in an extruder to form a fibrous protein composition;
(e) allowing the fibrous protein composition to exit the extruder through an extruder nozzle; wherein the water content of the aqueous protein composition is at least 50% by weight and wherein the fibrous protein composition is subjected to limited cooling so that it exits the extruder at a temperature of the composition at least equal to the boiling temperature of water in the first external environment. The protein content, based on the dry matter, is in a range from 15% by weight to 85% by weight and preferably in a range from 50% by weight to 80% by weight. The aqueous compositions are fed to an extruder, and the kneading and denaturing steps (b) and (c) are carried out in the extruder. The extruder has a length to diameter L/D ratio greater than 20, preferably greater than 30, for example it may be 40-50. The structured plant protein extrudate is subjected to a freezing process. It is also contemplated to heat the aqueous liquid to a temperature of 70° C. to 98° C. The liquid may contain flavorings but is preferably a broth. The plant protein extrudate contains 0.1-20% by weight of fat, preferably 0.2-10% by weight of fat. The product can be used to produce a meat-like, fibrous, textured plant protein

WO 2020/208104 A1 relates to a meat analog comprising a macrostructure of connected sheared fibers oriented substantially parallel to each other; and gaps located between the sheared fibers, wherein the macrostructure does not comprise meat and wherein the macrostructure comprises a plant protein. A fat and/or fat analog is injected into the vertical gaps such that the meat analog comprises a plurality of alternating visually distinct areas, the visually distinct areas comprising one or more first visually distinct areas comprising the fat and one or several second visually distinct areas comprising the plant protein. The vertical gaps can also be immersed in a fat solution such that the meat analog comprises a plurality of alternating visually distinct areas, the visually distinct areas being one or more first visually distinct areas comprising the fat and one or more second visually distinct areas comprising the plant protein. The macrostructure may comprise a texturized plant protein or micronized plant material, wherein the micronized plant material comprises at least one of the group consisting of hulls, fibers and mixtures thereof. The meat analog is shaped to resemble a marbled meat. The macrostructure should have a non-homogeneous structure. Also contemplated is an extrusion system for the production of a meat analog, the meat analog comprising a plant protein, the extrusion system comprising an extruder and a short nozzle; wherein the extruder is connectable to the short nozzle and configured to direct a material comprising a plant protein from the extruder to the short nozzle and through a fluid path extending through the short nozzle, wherein the short nozzle is configured to inject a fat or fat analog into the material such that the fat or fat analog is embedded but visually separated from the material comprising the plant protein when the fat or fat analog and the material leave the short nozzle. A method of extruding a meat analog is described, the following method steps to be applied: applying pressure to the meat analog with an extruder; and passing the meat analog through a short nozzle in a flow direction, the short nozzle being part of and/or connected to the extruder, and generating sheared fibers in the meat analog substantially perpendicular to the flow direction of the meat analog as the meat analog passes through the short nozzle. An addition of pea skin to the meat analog is also described, with the meat analog also being said to contain pea proteins or field bean proteins.

### Objects

The invention is based on the object of creating a foamed product of the specified type with predetermined functional properties.

Furthermore, the invention is based on the object of providing a device for producing such a product.

In addition, the invention is based on the object of proposing a method with which such a product can be produced.

Finally, the invention is based on the object of proposing a use according to the invention of such products.

### Achieving the Object Relating to the Product

This object is achieved according to the invention by a foamed, resilient, protein-based product with a dry matter fraction of 20-98% by weight, bound water fraction of 2-80% by weight and a partially to fully filled open pore structure, the ratio of the volume of fluid-filled pores open towards the product surface (OGP) to the total volume of open pores in the product is set in the range 0.05.

The present invention is based on a generic concept for the personalization of sustainably produced food systems made from plant protein and plant fibers, with special consideration of meat analogs. “High Moisture Extrusion Cooking” (HMEC) technology has become more and more established for the production of meat analogs, which already come quite close to prepared meat in terms of their texturing. Compact, fibrillar, meat-like structures can thus be achieved. However, this technology has so far been limited in terms of further improvement and defined adjustment of the sensory aspects (1) tenderness, (2) juiciness, (3) crispiness and (4) taste/aroma. The development of HMEC microfoaming (HMECF) of plant protein-based meat analogs, which is in its initial phase, has shown new possibilities for implementing next development steps in terms of improving the relevant sensory quality aspects (1)-(4).

This is where the product, process and device development according to the invention begins. In an invention made parallel to the further development according to the invention described at this point, mechanisms for the production of open-pored, foamed meat analogs could be identified. This paved the way for the prominent use of open-pored HMECF-based meat analogs to advance the improvement of their properties and personalization.

The adjustable filling of open foam pores in terms of degree of filling and filling fluid as well as the diverse possibilities of integrating a range of sensory, nutritional and health-related components into corresponding filling fluids led to the product, process and device developments according to the invention presented at this point. For filling open pores with characteristic diameters of approx. 10-500 microns, (a) capillary forces, (b) pore elastic relaxation after deformation, (c) infusion and (d) ultrasonically forced diffusion were identified as the main physical mechanisms and which are to be applied individually or in combination. The method steps considered relevant were developed based on these mechanisms and the devices suitable for carrying out the same were derived.

### Some Advantages

For the sensory optimization of a foamed meat analog texture, gas volume fractions of 5-10% already show a significant reduction in product firmness. Above 30% gas volume fraction, the aspect of increased soft rubberiness (“marshmallow consistency”) can become detectable. This is particularly true in the case of closed foam pores, since the trapped gas fraction expands again after compression when biting/chewing deformation is imposed. In the case of open pores, the gas in the pores is pushed out of the pores into the environment without any associated damping effect on the deformation process under deforming stress and is (partially) sucked in again when it relaxes. The gas inflow and outflow does not have any significant influence on the texture perception. If the open pores are filled with fluid, as the fluid viscosity increases, the outflow from open pores, which is delayed or impeded as a result, contributes to a firmer texture perception.

Furthermore, the sensory aspect of crunchiness is supported by open pores, since a critical elongation at break of the pore walls is more likely to be reached without a damping effect, such as that occurring through a non-escape gas cushion in a closed pore. When the pores are filled with fluids that have a low viscosity (up to approx. 100 mPas), “juiciness” is also perceived more strongly when the pores are deformed and the fluid escapes from open pores.

Thus, fluid-filled, open foam pore systems can be expected to have improved sensory properties in meat analogs with regard to tenderness, crispiness and juiciness.

### Further Inventive Configurations

Further inventive configurations are described in claims 2 to 19.

According to claim 2, the invention is characterized in that the volume fraction of filled open pores based on the total volume of all open and closed pores is between 0.05-1, preferably between 0.2-0.95, and is set for values ≥0.1 with an accuracy of +/−0.05.

For the food group of plant protein-based meat analogs that is particularly under consideration, a maximum of 50% gas volume fraction due to foaming is targeted in order to avoid excessively pronounced “rubberiness”. The pores are opened by means of pore opening technologies using the mechanisms (a) pore opening by setting a rapid drop in ambient pressure (Flash-Opening, FOP), (b) pore opening by splitting or peeling the product (CUT-Opening, COP), (c) pore opening by multiple needle penetration (Penetration-Opening, POP) or (d) pore opening by generating a secondary mixed flow (Mix-Opening, MOP) in the extruder cooling nozzle, adjustable usually between 10-60%. This means that in the case of the preferred contemplated meat analogs there is between 5-30% open pores.

According to claims 3 to 10, compositional aspects (water, protein, fiber contents) of the product foam structure are addressed, which on the one hand relate to the mechanical properties (structure strength, fibrillar structure) thereof, and on the other hand also to nutritional aspects (fiber, fat/oil fractions).

Claim 10 also refers to the possibility of carrying out the filling of the pores in the dried state of the foamed framework matrix with water contents ≤10% by weight, based on the total mass, in the semi-moist state with water contents between 10-40% by weight, or in the moist state of the foamed framework matrix, as it is present directly after HMECF production with water contents of between 40-70% by weight, based on the total mass. Although a drying step of the product after HMECF extrusion means additional production and energy expenditure, it is of interest with regard to product packaging, storage and shelf life aspects. Due to their high water content, meat analogs produced by HMECF can only be placed in food retailing under cold storage conditions. However, for the open-pored, foamed meat analogs, in the case of drying, there is the possibility with less complex packaging, to design the product storage under room temperature conditions and the functionalization by pore filling during reconstitution using the special structure-related instant reconstitution properties due to open pores, in the dried or partially dried state.

Claims 11 to 19 relate to the composition and properties of the pore-filling fluids with the associated diverse possibilities of introducing a wide range of sensory, nutritional and health-promoting functionalities into the products with filled pores via these pore-filling fluids.

### Some Advantages

Against this background, with the products according to the invention, filled with functional fluid fillings in the open product pores, a variety of problem solutions with regard to more personalized nutrition can be addressed in a significantly simplified manner. In addition to facilitating the manufacture of sensory and nutritionally customized products on an industrial production level, the end consumer will also be able to make corresponding individual optimizations under optimal convenience boundary conditions.

This will initially relate to the foamed, open-pored plant protein-based meat analogs according to the invention, but will not remain limited thereto, since the generic concept of the functional filling of open-pored food product systems on which the invention is based can be transferred to further new product developments.

### Achieving the Object Relating to the Method

This object is achieved according to claim 20 in that, for filling the open pores of the foamed product the four filling mechanisms are applied, individually or in combination, (a) filling by means of capillary forces (BK), (b) filling by means of elastic pore Relaxation (BE), (c) filling by infusion (BI), and (d) filling by diffusion (BD).

### Some Advantages

The mentioned filling mechanisms for open foam pores activated according to the invention can be combined in a simple manner with the HMEC extrusion technology and with the methods steps for opening the foam pores upstream of the filling of open pores, or can be integrated into a compact production process.

If, for example, pores are opened by static residual pressure release at the end of the extruder nozzle so that closed pores are opened towards the product surface, an associated elastic reverse deformation of the product matrix in the opened pore channel area can be used to suck in a fraction of pore-filling fluid, possibly supported by superimposed mechanical pressure deformation and elastic shape relaxation. The same applies to the combination of a pore opening via sudden vacuum application, which, when carried out in the immersion bath filled with pore-filling fluid, is followed by a subsequent suction of the fluid into open pores. Finally, pore opening by means of needle penetration can also be combined for filling using hollow needles. With regard to pore filling using hollow needles, it should be noted that this type of filling differs according to the invention from infusion methods known from the meat industry, such as those, e. g. used in the production of boiled ham, in that during needle penetration into a foam structure such as that in this case, the injected pore-filling fluid contributes to the rupture of pore walls as a result of the imposed filling pressure and thus further pores that are still closed are opened.

### Further Inventive Configurations

Claims 21 to 27 describe in detail the pore-filling methods by means of the mechanisms (a)-(d) in their respective procedural implementation. Aspects of adaptability to HMEC technology are highlighted.

### Achieving the Object Relating to the Device

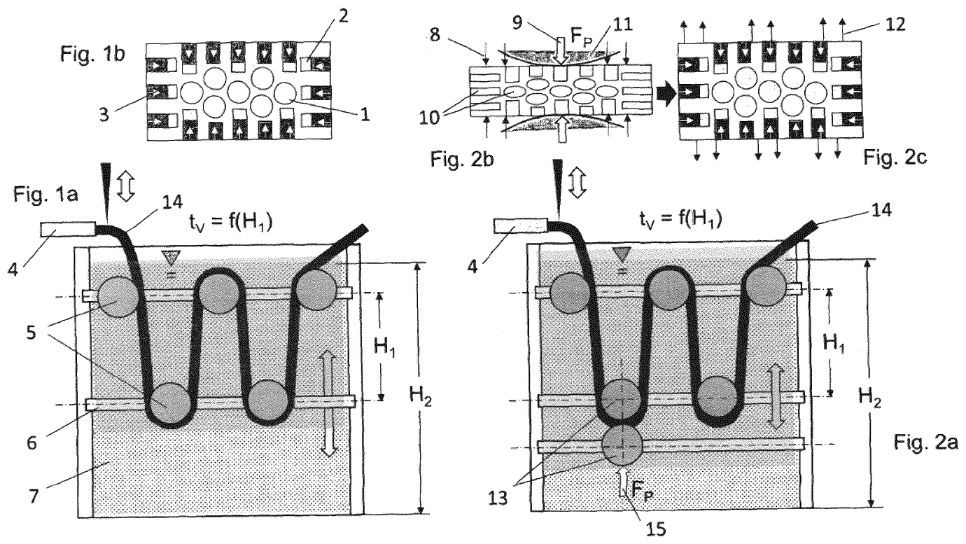

This object is achieved by claim 28 with reference to the four pore-filling mechanisms (a-d) differentiated according to the invention. The following description of the devices associated with these mechanisms is supplemented by FIGS. 1 to 5.

### Some Advantages

The devices for filling the open foam pores assigned to the method steps according to the invention are also technically compatible with HMEC technology and the various method steps downstream of the HMEC extruder, either individually or in combination, for adjustable pore opening. The inventive at least partial combinability of the devices for opening the foam pores with the devices for filling the open foam pores described in more detail below is of considerable advantage in an integrated design of the overall production process of tailor-made functionalized meat analogs, but not limited to these products.

### Further Inventive Configurations

According to claims 29 to 35, the pore-filling devices are described in detail based on the associated method steps using the pore-filling mechanisms (a)-(d) in their respective process engineering environment. Again, aspects of adaptability to HMEC technology are highlighted.

### Achieving the Object Regarding the Use

This object is achieved by claim 36 for foodstuff with specific sensory, nutritional and health-promoting properties for specific consumers or patient target groups.

### Some Advantages

Although there has been talk of personalized nutrition for years, practical implementation concepts only include larger target groups (e. g. those with certain intolerances or allergies or deficiency symptoms, pregnant women, infants or the elderly). This is due in particular to the additional effort in the production methods required in the industrial production of food products with a further reduction in the size of target groups down to individuals, and in the production and distribution logistics.

The concept according to the invention of filling open-pored foam systems with fluid systems that can be individually tailored with regard to sensory, nutritional and health aspects allows to reach a new milestone by the industrial production of open-pored product matrices with a downstream simplified functionalization step by pore filling with an assortment of fluids, flavorings and aromas (incl. spices), micro-nutrients (vitamins, trace elements) as well as health-promoting components (antioxidants) with simplified methods and reduced logistic effort.

### Further Inventive Configurations

Claims 37 and 38 underscore the above-mentioned advantageous possibilities of the product applications according to the invention and emphasize the special reference to plant protein-based meat analogs.

In the drawing, the invention is—partly schematically—described using exemplary embodiments. In the drawings:

FIG. 1a shows a device for filling open foam pores by means of capillary forces;

FIG. 1b shows a further detail in section:

FIG. 2a shows a device for filling open foam pores by means of elastic pore shape relaxation;

FIG. 2b shows a further detail in section;

FIG. 2c shows a further detail in section;

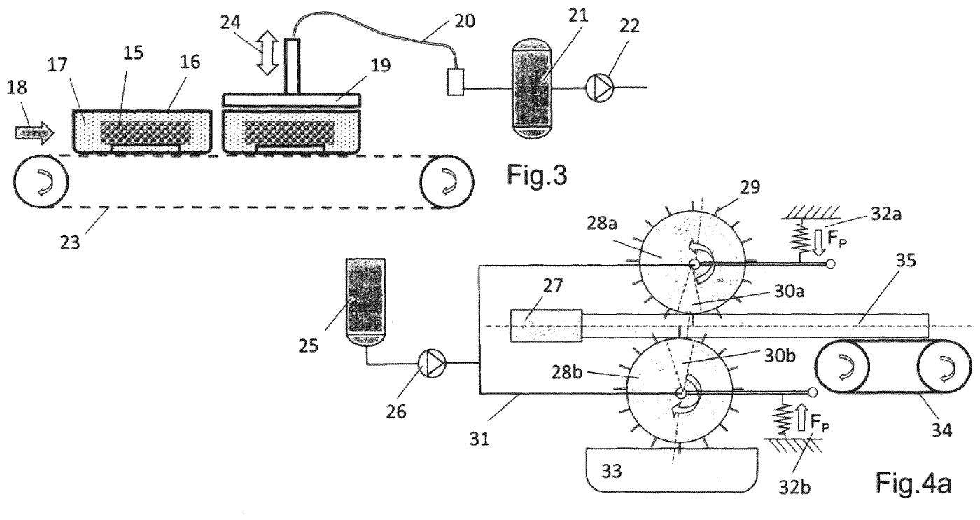

FIG. 3 shows a device for filling open foam pores by means of elastic pore shape relaxation after deformation by means of a partial vacuum;

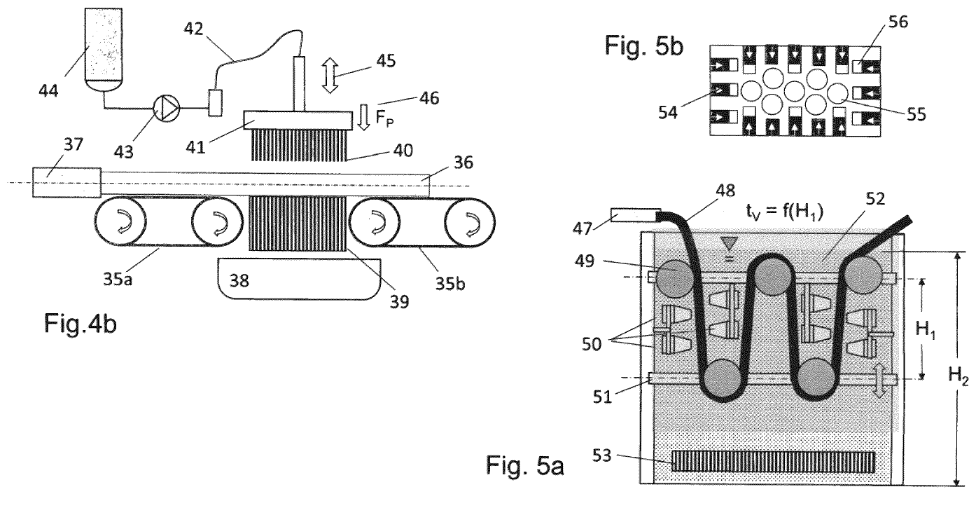

FIG. 4a shows a device for filling open foam pores by means of infusion via a hollow needle penetration and pore-filling roller system;

FIG. 4b shows a device for filling open foam pores by means of infusion via a hollow needle penetration and pore-filling punch system; and

FIGS. 5a and 5b show a device for filling open foam pores by means of ultrasonically forced diffusion.

FIG. 1a shows a device arrangement with a HMEC extruder nozzle 4 and deflection guide rollers 5 for an extrudate strand. Height-adjustable brackets for lower rows of deflection rollers are denoted by 6. Numeral 7 denotes a pore-filling fluid. The height adjustment range of the adjustable brackets 6 is shown schematically with H₁, while H₂ is the total height of the pore-filling fluid bath.

In FIG. 1b, reference numeral 1 denotes closed foam pores, while 2 are open foam pores. An inflowing pore-filling fluid is indicated schematically at 3.

FIG. 2a shows a schematic representation of a pore-filling device by means of elastic pore shape relaxation after extrudate strand passage 14 between pressure rollers 13.

In FIG. 2b, reference numeral 8 denotes a direction of compression between pressure rollers 11 by means of a pressure force 9, which acts on the open and closed pores 10 of an extrudate strand.

In FIG. 2c, reference numeral 12 denotes the direction of reverse expansion of an extrudate strand under shape relaxation after exiting the pressure roller gap.

FIG. 3 shows a device for filling open foam pores by means of elastic pore relaxation after deformation by applying a partial vacuum. Reference numeral 15 denotes an extrudate strand part, while 16 is a half-shell for accommodating strand parts for vacuum treatments. 17 is the pore-filling fluid and 18 denotes the conveying direction. 19 denotes a movable cover device for the sealing closure of the lower half-shell with product parts. Reference numeral 20 denotes a vacuum hose connected to a vacuum tank 21. 22 denotes a vacuum pump, and 23 denotes a conveyor belt. The direction of rotation and thus the conveying direction of the conveyor belt 23 results from the arrows at the reversing stations of the conveyor belt 23. The reference number 24 denotes a periodic movement of the cover construction for the vacuumizing trays 16.

FIG. 4a shows a device for filling open foam pores by means of infusion via hollow needles of a penetration and pore-filling roller system. 25 denotes a storage tank for a pore-filling fluid and 26 denotes a piston metering pump, while 27 represents the end of the HMEC extruder cooling nozzle. Shown at 28a is an upper hollow needle penetration and pore-filling roller, while 28b illustrates a lower hollow needle penetration and pore-filling roller. A penetration and filling hollow needle is denoted by 29, while 30a denotes a metering segment of the upper pore-filling roller. 30b is a metering segment of the lower pore-filling roller. Illustrated at 31 is a pore-filling fluid feed line to the hollow needle penetration and pore-filling rollers. Reference numeral 32a denotes a pressure pressing device for the upper hollow needle penetrating and pore-filling roller 28a. Fₚ denotes the direction of pressure, while 32b illustrates a pressure pressing device for the lower hollow needle penetration and pore-filling roller. A collection container for pore-filling fluid is denoted by 33. Shown at 34 is a conveyor belt for the removal of the treated products. The conveying direction is apparent from the arrows of the deflection stations.

FIG. 4b shows a device for filling open foam pores by means of infusion via a hollow-needle penetration and pore-filling punch system. Conveyor belts for the extrudate strand are shown at 35a and 35b, while 36 denotes an extrudate strand which is indicated by way of example. An extruder nozzle outlet is denoted by 37, while 38 denotes a collecting container for pore-filling fluid. At 39 a perforated sheet metal block for accommodating penetration and pore-filling hollow needles is indicated schematically, while 40 represents a penetration and pore-filling hollow needle punch equipped with several penetration and pore-filling hollow needles. 41 illustrates a pore-filling fluid supply for the penetration and pore filling hollow needle punch. 42 denotes a pressure hose supply for pore-filling fluid, while 43 is a pore-filling fluid metering pump fed from the pore-filling fluid container 44. The direction of movement of the penetration and pore-filling hollow needle punch is shown with reference number 45, while a hydraulic penetration pressure booster is indicated schematically with 46.

FIG. 5a shows a device for filling open foam pores by means of ultrasound-forced diffusion, an extruder nozzle outlet is shown at 47, while 48 is an HMEC extrudate strand. Deflection/guide rollers for the extrudate strand are shown at 49. Ultrasonic sonotrodes are indicated schematically at 50, while 51 illustrates a height-adjustable suspension of the lower deflection rollers for the extrudate strand 48. The extrudate residence time in the heated ultrasonic immersion bath can be set by adjusting the height. A pore-filling fluid is indicated by reference numeral 52, while 53 is a heating register for heating pore-filling fluid.

The height adjustability of the lower deflection rollers is denoted by H₁, while H₂ indicates the overall height of the pore-filling fluid bath.

In FIG. 5b, 54 indicates the diffusion direction in open foam pores, while 56 denotes open foam pores and 55 denotes closed foam pores.

The features described in the claims and in the description and evident from the drawing can be essential for the implementation of the invention both individually and in any combination.

### LIST OF REFERENCE NUMERALS

1 foam pores, closed
2 foam pores, open
3 pore fill fluid, inflowing
4 HMEC extruder nozzle
5 deflection guide roller for extrudate strand
6 bracket, height adjustable
7 pore-filling fluid
8 direction of compression between pressure rollers
9 roller pressure force on the extrudate strand
10 pores, deformed/compressed (open/closed)
11 pressure roller
12 extrudate strand expansion/relaxation at pressure roller exit
13 pressure roller
14 extrudate strand
15 extrudate strand part
16 half-shell to accommodate strand parts for vacuum treatment
17 pore-filling fluid
18 conveying direction
19 movable cover device for the sealing closure of the lower half-shell with product parts
20 vacuum hose line
21 vacuum tank
22 vacuum pump
23 conveyor belt
24 periodic movement of the cover construction for vacuumizing tray
25 storage tank for pore-filling fluid
26 piston metering pump
27 end of the HMEC extruder cooling nozzle
28a upper hollow needle penetration and pore-filling roller
28b lower hollow needle penetration and pore-filling roller
29 penetration and filling hollow needle
30a metering segment of the upper pore-filling roller
30b metering segment of the lower pore-filling roller
31 pore-filling fluid supply to hollow needle penetration and pore-filling rollers
32a top roller pressure device
32b bottom roller pressure device
33 pore-filling fluid collection vessel
34 conveyor belt
35a extrudate strand conveyor belt
35b extrudate strand conveyor belt
36 HMEC extrudate strand
37 extruder nozzle outlet
38 collection container for pore-filling fluid
39 perforated sheet metal block for accommodating penetration and pore-filling hollow needles
40 penetration and pore filling hollow needle punch
41 pore-filling fluid supply to penetration and pore-filling hollow needle punches
42 pressure hose supply line for pore-filling fluid
43 dosing pump
44 pore-filling fluid container
45 direction of movement of the penetration and pore filling hollow needle punch
46 hydraulic penetration pressure booster
47 extruder nozzle outlet
48 HMEC extrudate strand
49 deflection/guide roller for extrudate strand
50 ultrasonic sonotrodes
51 height-adjustable suspension of the lower deflection rollers to adjust the extrudate strand residence time in heated ultrasonic immersion bath
52 pore-filling fluid
53 heater register for pore-filling fluid heating
54 diffusion direction in foam pores, open
55 foam pores, closed
56 foam pores, open
H₁ height adjustment range of the lower deflection rollers
H₂ total height of the pore-filling fluid bath
t_{V} residence time of the extrudate strand in the pore-filling fluid bath
Fₚ direction of pressure

### LITERATURE REFERENCES

/1/ P. J. Hailing & P. Walstra (1981) Protein-stabilized foams and emulsions, C R C Critical Reviews in Food Sci. & Nutrition, 15:2, 155203, DOI: 10.1080/1040839810952 7315
/2/ L. Forny, A. Marabi, S. Palzer (2009); Wetting, disintegration and dissolution of agglomerated water soluble powder; June 2009 Conference: 9th International Symposium on Agglomeration and 4th International Granulation Workshop; Sheffield; Volume: paper 64
/3/ Moore G. (1994) Snack food extrusion. In: Frame N. D. (eds) The Technology of Extrusion Cooking. Springer, Boston, MA. https://doi.org/10.1007/978-1-4615-2135-8_4; Print ISBN978-1-4613-5891-6
/4/ V. Lammersl, A. Morant, J. Wemmer, E. Windhab (2017); High-pressure foaming properties of carbon dioxide-saturated emulsions; Rheol Acta (2017) 56:841-850
/5/ E. Windhab, V. Lammers (2017); Patent: Aufgeschäumtes teigbasiertes Lebensmittelprodukt sowie Vorrichtung und Verfahren zur Herstellung des aufgeschäumten teigbasierten Lebensmittelprodukts; Patent Application No. DE 10 2016 111 518 A1
Albert Schweitzer Foundation: Vegane GroBverpflegung—ein Leitfaden, 2. edition, as of May 2017
WO 2012/158023 A1
WO 2020/208104 A1

1.-38. (canceled)

39. A foamed, resilient, protein-based product with a dry matter fraction of 20-98% by weight, bound water fraction of 2-80% by weight and a partially to fully filled open pore structure, the ratio of the volume of fluid-filled pores open towards the product surface (OGP) to the total volume of open pores (OP) in the product is set in the range 0.05-1.00, for values of this ratio of ≥0.1 with an accuracy of +/−0.05, and the pore-filling fluid is preferably enriched with components which add a sensory and/or nutritional and/or flavor and/or pharmaceutical function to the product.

40. The product according to claim 39, wherein the volume fraction of filled open pores based on the total volume of all open and closed pores is preferably between 0.2-0.95, and is set for values ≥0.1 with an accuracy of +/−0.05.

41. The product according to claim 39, wherein the total fraction of pores (=porosity) is also set between 0.1 and 0.95 with an accuracy of +/−0.05.

42. The product according to claim 39, wherein the product has a protein fraction of 10-95% by weight, based on its dry matter.

43. The product according to claim 39, wherein the protein fraction consists of 0-100% plant protein.

44. The product according to claim 39, wherein the protein in the product is present in a partially to fully denatured form and preferably has a fibrillar structure, more preferably an oriented fibrillar structure.

45. The product according to claim 39, wherein the product includes a plant fiber fraction of 0.5-20% by weight, based on the dry matter.

46. The product according to claim 39, wherein the product includes a fraction of fats or oils of 0.1-15% by weight, based on the dry matter.

47. The product according to claim 39, wherein this product corresponds to a plant protein-based, foamed meat analog, which is produced using High Moisture Extrusion Cooking (HMEC) technology and the open pores of which are filled with a fluid to a set fraction, which adds an additional sensory and/or nutritional and/or pharmaceutical function to the product.

48. The product according to claim 39, wherein in the filled state said product as a dried, foamed framework matrix is set to water contents of ≤10% by weight, based on the total mass, as a semi-moist, foamed framework matrix to water contents of between 10-40% by weight, based on the total mass, or as a moist, foamed framework matrix to water contents of between 40-70% by weight, based on the total mass.

49. The product according to claim 39, wherein the pore-filling fluid in the filled state is set to a zero dynamic viscosity of ≤1 Pas and wetting properties with respect to the material forming the pore walls.

50. The product according to claim 39, wherein the pore-filling fluid retains its fluid character after the pores have been filled.

51. The product according to claim 39, wherein the pore-filling fluid forms a yield point after the pores have been filled.

52. The product according to claim 39, wherein the pore-filling fluid is enriched with flavorings or aromas which are sensorily relevant for human consumption, or a combination thereof.

53. The product according to claim 39, wherein the pore-filling fluid is enriched with nutritive components relevant to human diet.

54. The product according to claim 39, wherein the pore-filling fluid is enriched with pharmaceutical active ingredient components which are indicated for certain disease prophylaxis or the treatment of certain diseases.

55. The product according to claim 39, wherein the pore-filling fluid is a multi-phase system with emulsion, multiple emulsion or suspension character.

56. The product according to claim 39, wherein the pore-filling fluid is a multi-phase system with an emulsion, multiple emulsion or suspension character and includes or encapsulates different sensory or nutritive functionalizing substance components in the individual phases thereof.

57. The product according to claim 39, wherein the pore-filling fluid consists of soups, sauces, dressings, drinks or a fat melt.

58. A method for filling open pores for products according to claim 39, wherein for filling the open pores of the foamed product the four filling mechanisms are applied, individually or in combination: (a) filling by means of capillary forces (BK), (b) filling by means of elastic pore Relaxation (BE), (c) filling by infusion (BI), and (d) filling by diffusion (BD), the degree of filling of the open pores being set by adapting the driving forces activated in a.)-d.).

59. The method according to claim 58, wherein the four filling mechanisms according to claim 58 are used individually or in combination spatially and temporally directly downstream of the HMEC technology.

60. The method according to claim 58, wherein the (a) filling of open pores by means of capillary forces via direct contacting of an HMEC extruded product strand with open pore fractions occurs in a filling fluid bath, through which the extrudate strand is continuously moved below the fluid level.

61. The method according to claim 58, wherein the (b) filling of open pores by means of the mechanism of elastic pore relaxation occurs via the imposition of a deformation constricting the open pores and the resulting elastic reverse deformation of the same in a filling fluid bath, the suction pressure generated by the elastic relaxation of the open pores causing the pore filling.

62. The method according to claim 58, wherein the force for pore deformation to be applied for (b) filling the open pores by means of the mechanism of elastic pore relaxation occurs continuously via compressive stress on the HMEC extrudate strand between two roller elements in the filling fluid bath.

63. The method according to claim 58, wherein the force to be applied for pore deformation for (b) filling the open pores by means of the mechanism of elastic pore relaxation occurs quasi-continuously via a partial vacuum stress on cut HMEC extrudate strand parts in the hermetically encapsulated filling fluid bath.

64. The method according to claim 58, wherein (c) the filling of open pores occurs by means of the infusion mechanism, with the filling fluid being injected into the foamed pores via injection needles and both previously open pores and pores newly created by the needle punctures or connection channels between previously closed pores are filled.

65. The method according to claim 58, wherein the (d) filling of open pores occurs by means of diffusion, the filling fluid being heated to temperatures below its boiling point or below a critical change temperature for its sensory or nutritive functionality in the immersion bath, and flowing over the foamed HMEC extrudate strand with open foam pores that is moving continuously through said immersion bath, as well as an increase in the diffusion rate and the opening of further initially closed pores is effected via the imposition of ultrasonic waves at a frequency in the range of 10-50 kHz and a volumetric ultrasonic power of 0.1-0.5 kW/liter.

66. A device for filling open pores for products according to claim 39, wherein the set partial to complete filling of pores open towards the product surface occurs using the four filling mechanisms (a) filling by means of capillary forces (BK) in an immersion bath device, (b) filling by means of elastic pore relaxation (BE) in an immersion bath device with (i) pressure roller passage or (ii) vacuumizing unit, (c) filling by infusion (BI) by means of hollow needle penetration and filling device, and (d) filling by diffusion (BD) in an immersion bath with an integrated ultrasonic treatment device, individually or in combination, and directly spatially and temporally downstream of the HMEC extrusion device.

67. The device according to claim 66, wherein a filling fluid bath with a roller arrangement is attached to the HMEC extruder nozzle for the continuous guidance of the elastically flexible extrudate strand emerging from the extruder nozzle through the immersion bath, the roller arrangement and immersion bath being designed in such a way that a residence time of the extrudate strand immersed in the pore-filling fluid is between 5-10 seconds, adjustable to the extrusion speed or extrudate mass flow.

68. The device according to claim 66, wherein an adjustment device for the vertical distance between successive extrudate strand deflection rollers arranged in the immersion bath is integrated, with which the immersion length of the HMEC extrudate strand in the filling fluid bath is dynamically adapted to its nozzle outlet speed or extrudate mass flow so as to ensure a predetermined residence time in the immersion bath.

69. The device according to claim 66, wherein two rollers controlled by pressure force or pressure deformation are arranged in the filling fluid bath for the imposition of a temporary pressure deformation on the HMEC extrudate strand and a strand relaxation residence section below the fluid level is integrated downstream by means of further rollers for guiding the extrudate strand.

70. The device according to claim 66, wherein a cutting device for the extrudate strand is arranged directly downstream of the extruder nozzle outlet and for further processing of the cut extrudate strand parts a hermetically sealable filling fluid bath is integrated, in which a partial vacuum in the range of 10-100 mbar for 5-30 s is provided by means of a vacuum pump and vacuum container as well as subsequent ventilation and vacuum relaxation.

71. The device according to claim 66, wherein two rotatably suspended needle rollers, equipped with hollow needles with inner diameters between 0.3-5 mm, are arranged in such a way that the strip-shaped extruded product is guided between them and the needle penetration depth, depending on the product shape, can be set between 1-20 mm and the puncture number density can be set between 1-49/cm², with a piston metering pump being arranged to transport the pore-filling fluid, which fills the interior of the needle roller with pore-filling fluid via a hollow shaft and from there supplies the hollow needles penetrating the extrudate strand with pore-filling fluid for injection.

72. The device according to claim 66, wherein a punch equipped with hollow needles with hollow needle inner diameters of between 0.3-5 mm is arranged via a perforated plate with a hole arrangement geometrically matched to the needle arrangement on the punch, and a roller-based transport device for the strip-shaped extrudate continuously guided over said perforated plate therewith and a piston pump are integrated in order to implement volumetrically controlled injection of the pore-filling fluid into the extrudate strand with periodic lowering of the hollow-needle punch.

73. The device according to claim 66, wherein an immersion bath with a temperature control device for setting temperatures below the pore-filling fluid boiling temperature or a critical change temperature of the sensory or nutritive functionality of the same are arranged, the walls of the immersion bath being equipped with ultrasonic sonotrodes which implement US frequencies in the range of 10-50 kHz at a volumetric ultrasonic power of 0.1-0.5 kW/liter to increase the diffusion rate of pore-filling fluid into the open extrudate pores.

74. A use of a product according to claim 39 as food with specific sensory, nutritional and health-promoting properties for specific consumers or patient target groups.

75. The use according to claim 74, wherein the product is designed as a personalized plant-protein-based meat analog in which (i) meat-relevant taste and aroma components or (ii) nutritionally relevant B vitamins such as B12 and other vitamins and minerals such as bioavailable iron compounds or (iii) combinations of (i) and (ii) are incorporated in the pore-filling fluid.

76. The use according to claim 75, wherein the foamed product containing open pores and designed as a plant protein-based meat analog is subjected by the consumer himself/herself to a personalized filling of the open pores by placing and repeated periodically compressing in an individually composed fluid phase tailored to personal sensory, nutritional and health-promoting functions, the latter optionally corresponding to one of the food categories soups, sauces, dressings/marinades, drinks or melts.
